(19) United States
(12) Patent Application Publication
(10) Pub. No.: US 2024/0424263 A1
(43) Pub. Date: Dec. 26, 2024

(54) SUPPORT-FRAME-CENTERED CATHETER FOR CHEMICAL ABLATION

(71) Applicant: Medtronic Ireland Manufacturing Unlimited Company, Dublin (IE)

(72) Inventors: Jaclyn N. Kawwas, Philadelphia, PA (US); Jonathan Ashley Cope, Santa Rosa, CA (US); Kelsey Sandquist, Cotati, CA (US); Mikayla Novak, Rohnert Park, CA (US); Enda Carter, Galway (IE); Gerry Oliver McCaffrey, Abbeyknockmoy (IE)

(21) Appl. No.: 18/698,666

(22) PCT Filed: Oct. 25, 2022
(86) PCT No.: PCT/EP2022/079788

§ 371 Date: Apr. 4, 2024

Related U.S. Application Data
(60) Provisional application No. 63/273,669, filed on Oct. 29, 2021.

(51) Int. Cl. A61M 25/04; A61B 18/00; A61M 25/00

(52) U.S. Cl. CPC A61M 25/04; A61B 2018/00214; A61B 2018/00577; A61M 2025/0085

(57) ABSTRACT

In some examples, a medical system for delivering a chemical agent into a perivasculature of a patient includes a catheter (102) defining a central longitudinal axis; and a neuromodulation assembly at a distal portion of the catheter, wherein the neuromodulation assembly includes one or more fluid-delivery needles (120) configured to extend radially outward from the central longitudinal axis at a distal portion of the catheter; and a radially expandable support frame (124) positioned both proximal and distal to the one or more fluid-delivery needles, the radially expandable support frame configured to contact a vessel wall of a vessel (118) of a patient to approximately radially center the distal portion of the catheter within the vessel and to distribute an applied pressure between the neuromodulation assembly and the vessel wall.

### TECHNICAL FIELD

The present technology is related to chemical neuromodulation.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

### SUMMARY

The present technology is directed to devices, systems, and techniques for neuromodulation, such as renal neuromodulation, using needles. More specifically, the present disclosure describes a medical system and associated techniques for delivering a chemical agent (e.g., an ablation fluid), into perivasculature tissue of a patient, such as renal perivasculature tissue. Although renal neuromodulation is primarily described herein, devices, systems, and techniques described herein may be applied to other types of neuromodulation, such as neuromodulation performed on nerves other than the renal nerves, at sites other than within a renal vessel, or both. In general, the devices, systems, and techniques described herein may be used to perform neuromodulation from within any suitable anatomical lumen that has nerves adjacent to the anatomical lumen.

In some examples, the medical system includes a neuromodulation catheter, a distal portion of which includes a neuromodulation assembly having a plurality of therapeutic elements, e.g., fluid-delivery needles, and a radially expandable support frame positioned both proximal and distal to the therapeutic elements. The support frame may include an expandable basket-like, cage-like, and/or stent-like structure.

In an expanded or deployed configuration, the expandable support frame is configured to contact a vessel wall of the vessel both proximal and distal to the locations at which the plurality of therapeutic elements are configured to extend at least partially through the renal vessel wall to deliver the chemical agent. By contacting the vessel wall at these locations, the expandable support frame helps approximately center the distal portion of the catheter within the vessel and helps retain the distal catheter portion in position relative to the vessel wall during a neuromodulation treatment. In some examples, the neuromodulation catheter further includes a corresponding guide tube surrounding or housing each needle, e.g., extending radially outward from the catheter. In some such examples, the expandable support frame is configured to help protect the vessel wall by distributing, over a larger area, a pressure applied by contact between the neuromodulation assembly and the vessel wall.

Also disclosed herein is a medical system for delivering a chemical agent into a perivasculature of a patient includes a catheter defining a central longitudinal axis; and a neuromodulation assembly at a distal portion of the catheter, wherein the neuromodulation assembly includes one or more fluid-delivery needles configured to extend radially outward from the central longitudinal axis at a distal portion of the catheter; and a radially expandable support frame positioned both proximal and distal to the one or more fluid-delivery needles, the radially expandable support frame configured to contact a vessel wall of a vessel of a patient to approximately radially center the distal portion of the catheter within the vessel and to distribute an applied pressure between the neuromodulation assembly and the vessel wall.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout.

FIG. 1 is a schematic illustration of an example medical system that includes a neuromodulation catheter.

FIG. 2 is a conceptual diagram illustrating a technique for using the neuromodulation catheter of FIG. 1 to access a renal vessel of a patient and modulate renal nerves of the patient.

FIG. 3 is a conceptual diagram illustrating an example sympathetic nervous system (SNS) of a patient, and in particular, a mode of communication between the patient's brain and the patient's body via the SNS.

FIG. 4 is an enlarged anatomic view of nerves innervating a left kidney to form the renal plexus surrounding the left renal vessel of a patient.

FIGS. 5 and 6 are anatomic and conceptual diagrams, respectively, of a human body depicting neural efferent and afferent communication between the brain and kidneys of the human body.

FIGS. 7 and 8 are anatomic diagrams of an arterial vasculature and a venous vasculature, respectively, of a human body.

FIG. 9 is a conceptual diagram of an example of the neuromodulation catheter of FIG. 1, in which a distal portion of the catheter is positioned within a renal vessel.

FIG. 10 is a schematic cross-sectional view of the neuromodulation catheter of FIG. 9 taken along line A-A, with the therapeutic elements in a delivery configuration.

FIG. 11 is a schematic cross-sectional view of the neuromodulation catheter of FIG. 9 taken along line A-A, with the therapeutic elements in a deployed configuration.

FIGS. 12A and 12B are perspective views of a first example neuromodulation assembly of the catheter of FIG. 1.

FIG. 12C is a perspective view of an example ring structure of the neuromodulation assembly of FIGS. 12A and 12B.

FIG. 12D is a perspective view of an example of the neuromodulation assembly of FIGS. 12A and 12B without the proximal and distal ring structures.

FIG. 13A is a perspective view of a second example neuromodulation assembly of the catheter of FIG. 1.

FIG. 13B is a perspective view of another example of the neuromodulation assembly of FIG. 13A.

FIGS. 14A and 14B are perspective views of a third example neuromodulation assembly of the catheter of FIG. 1.

FIG. 14C is a perspective view of an example of the neuromodulation assembly of FIGS. 14A and 14B with a modified tubular structure.

FIG. 15A is a perspective view of a fourth example neuromodulation assembly of the catheter of FIG. 1.

FIG. 15B is a cross-sectional view of a portion of the catheter of FIG. 15A, the cross-section taken through line B-B of FIG. 15A.

FIGS. 16A and 16B are perspective views of a fifth example neuromodulation assembly of the catheter of FIG. 1.

FIG. 17 is a flow diagram illustrating an example technique of performing neuromodulation of renal nerves using the medical system of FIG. 1.

### DETAILED DESCRIPTION

The present technology is directed to devices, systems, and methods for neuromodulation, such as renal neuromodulation, using chemical agents. Although the following examples will be described primarily with respect to renal neuromodulation, a person having ordinary skill in the art will understand that the devices, systems, and methods described herein may be used for neuromodulation at any suitable intravascular location within a body of a patient. As used herein, the terms “proximal” and “distal” define relative positions or directions with respect to a treating clinician or clinician's control device (e.g., a handle assembly). For instance, “proximal” and “proximally” can refer to a position near or in a direction toward the clinician or clinician's control device, whereas “distal” or “distally” can refer to a position distant from or in a direction away from the clinician or clinician's control device.

Renal neuromodulation, such as renal denervation, may be accomplished using one or more of a variety of treatment modalities, including radio frequency (RF) energy, microwave energy, ultrasound energy, a chemical agent, or the like. When using a chemical agent, a neuromodulation catheter may be delivered to a renal vessel, such as a renal artery or renal vein, of a patient. The neuromodulation catheter may include at least one port or needle through which the chemical agent is delivered. The chemical agent may be selected to modulate activity of one or more renal nerves adjacent to the renal vessel in which the neuromodulation catheter is positioned. For example, the chemical agent may be a neurotoxic chemical selected to chemically ablate the one or more renal nerves near the renal vessel.

In accordance with techniques of this disclosure, a distal portion of a neuromodulation catheter includes a plurality of therapeutic elements and a radially expandable support frame positioned both proximal and distal to a plurality of therapeutic elements. The plurality of therapeutic elements may be arranged around an outer perimeter (e.g., referred to herein as a circumference, though the catheter may have a cross-section other than a circle in other examples) of the distal portion of the catheter. For example, the neuromodulation catheter may include three or more therapeutic elements arranged around a circumference of the distal portion of the catheter. In some implementations, the therapeutic elements may each include a needle (also referred to herein as a “microneedle”). The needle(s) are configured to deploy or extend radially outward, relative to a longitudinal axis of the neuromodulation catheter, to pierce a wall of the renal vessel and deliver a chemical agent.

In an expanded or deployed configuration, the expandable support frame is configured to contact the renal vessel wall both proximal and distal to the locations at which the plurality of therapeutic elements are configured to extend through the vessel wall. By contacting the vessel wall at these locations, the expandable support frame increases a contact area between the catheter and the vessel wall, e.g., compared to examples in which the needles or guide tubes for the needles are the only structures that contact the vessel wall. This may help approximately center the distal portion of the catheter within the renal vessel, help retain the distal catheter portion in position relative to the renal vessel wall during the neuromodulation treatment, and/or help protect the vessel wall by redistributing an applied pressure between the therapeutic elements and the vessel wall.

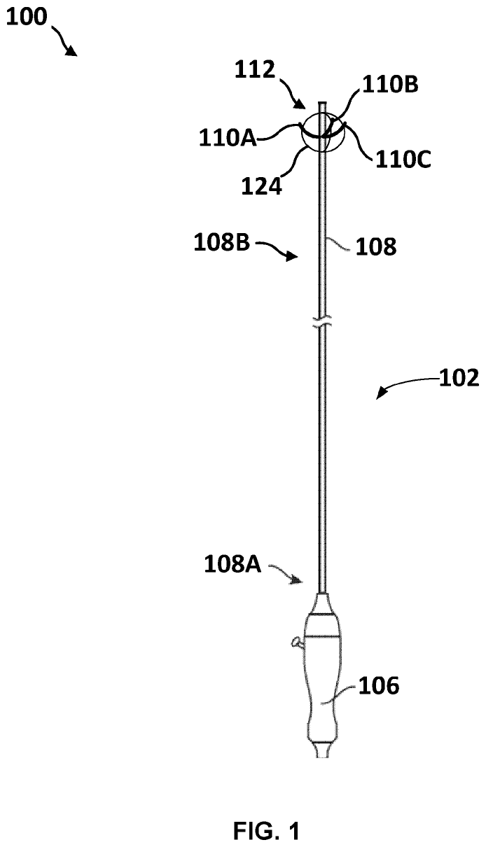

FIG. 1 is a partially schematic illustration of a medical system 100 (“system 100”) configured in accordance with some examples of the present technology. As shown in FIG. 1, system 100 includes at least a neuromodulation catheter 102. Neuromodulation catheter 102 includes a handle 106 and an elongated shaft 108 attached to the handle 106. The elongated shaft 108 may include a proximal portion 108A and a distal portion 108B. Elongated shaft 108 may have any suitable outer diameter (e.g., cross-sectional width), and the diameter can be constant along the length of elongated shaft 108 or may vary along the length of elongated shaft 108. In some examples, elongated shaft 108 can be about 2 French (Fr), about 3 Fr, about 4 Fr, about 5 Fr, about 6 Fr, about 7 Fr, or any other suitable size.

As shown in FIG. 1, distal portion 108B of neuromodulation catheter 102 includes a neuromodulation assembly 112 that includes a plurality of therapeutic elements 110A-110C (collectively, “therapeutic elements 110”). Therapeutic elements 110 may be positioned around (e.g., distributed around) a circumference of distal portion 108B. Although distal portion 108B is shown in FIG. 1 as including three therapeutic elements 110 positioned around a circumference of distal portion 108B at a single longitudinal position along elongated shaft 108, in other examples, distal portion 108B may include any number of therapeutic elements 110. For example, distal portion 108B may include two, three, four, or more therapeutic elements 110 positioned around a circumference of distal portion 108B at a common longitudinal position. In other examples, distal portion 108B may include positioned two, three, four, or more therapeutic elements 110 positioned around a circumference of distal portion 108B at each of multiple longitudinal positions along distal portion 108B. In examples in which distal portion 108B includes therapeutic elements 110 positioned at different longitudinal positions, each longitudinal position may include one or more therapeutic elements 110, and each longitudinal position may include the same number of therapeutic elements 110, or one longitudinal position may include a different number of therapeutic elements 110 than one or more other longitudinal positions.

Distal portion 108B of elongated shaft 108, including neuromodulation assembly 112, is configured to be moved within an anatomical lumen of a human patient to locate therapeutic elements 110 at a target site within or otherwise proximate to the anatomical lumen. For example, elongated shaft 108 may be configured to position therapeutic elements 110 within a blood vessel, a ureter, a duct, an airway, or another naturally occurring lumen within the human body. The examples described herein focus on the anatomical lumen being a blood vessel, such as a renal vessel, but it will be understood that similar techniques may be used with other anatomical lumens. In certain examples, intravascular delivery of the therapeutic elements 110 includes percutaneously inserting a guidewire (not shown) into a vessel of a patient and moving elongated shaft 108 and/or therapeutic elements 110 along the guidewire until therapeutic elements 110 reach a target treatment site (e.g., within a renal artery). For example, distal portion 108B of elongated shaft 108 may define a passageway for engaging the guidewire for delivery of therapeutic elements 110 using over-the-wire (OTW) or rapid-exchange (RX) techniques. In other examples, neuromodulation catheter 102 can be a steerable or non-steerable device configured for use without a guidewire. In still other examples, neuromodulation catheter 102 can be configured for delivery within an inner lumen of a guide catheter or sheath (not shown), or other guide device.

Once at the target site, therapeutic elements 110 can be configured to deliver therapy, such as RF energy, microwave energy, ultrasound energy, a chemical agent, or the like, to provide or facilitate neuromodulation therapy at the target site. For ease of description, the techniques of this disclosure are described with respect to examples in which the therapy includes a chemical agent, such as a neurotoxic chemical, and in which therapeutic elements 110 include needles (e.g., microneedles). It is to be understood, however, that therapeutic elements 110 may include elements or structures configured to deliver other types of therapy. For example, therapeutic elements 110 may include needle-electrodes configured to deliver RF energy for RF ablation of nerves near the blood vessel in which neuromodulation catheter 102 is positioned.

In examples in which neuromodulation catheter 102 is configured to deliver a chemical agent, therapeutic elements 110 may include deployable needles, configured to extend radially outward from distal shaft portion 108B and at least partially pierce a wall of the blood vessel (e.g., artery, vein, etc.) in which distal shaft portion 108B is positioned. Needles 110 may extend to and/or through the intima, media, and/or adventitia of the vessel wall and deliver the chemical agent to the adventitia and/or peri-adventitia, in which renal nerves are located. By having therapeutic elements 110 distributed around a circumference of distal shaft portion 108B, neuromodulation catheter 102 may be used to deliver the chemical agent around a circumference of the blood vessel in which distal shaft portion 108B is positioned. Again, while a “circumference” of the blood vessel is generally referred to herein, the blood vessel may not necessarily be perfectly circular in cross-section, and instead, may define any suitable cross-sectional geometry.

In accordance with techniques of this disclosure, neuromodulation assembly 112 includes a radially expandable support frame 124 that positioned both proximal and distal to therapeutic elements 110. Support frame 112 may include one or more expandable cage-like or stent-like elements that provide a number of benefits over neuromodulation assemblies that do not include a support frame of this nature. For instance, support frame 124 is configured to expand radially outward in the vicinity of therapeutic elements 110 so as to approximately center distal shaft portion 108B within a target vessel. In this way, therapeutic elements 110 may be more accurately deployed and positioned around the circumference of the interior surface of the vessel, and/or more accurately deployed a desired depth into and/or through the vessel wall.

As another example, and as detailed further below, in examples in which therapeutic elements 110 include a plurality of micro-needles, the therapeutic elements may often further include a corresponding plurality of guide tubes to surround and house the respective micro-needles. In some such examples, a radially outward portion of each guide tube may be operatively coupled to, or integrated with, a radially inward portion of the expandable support frame. In such configurations, expandable support frame 124 is configured to redistribute an applied pressure that might otherwise result from contact between the outward portions of the guide tubes and the vessel wall, thereby reducing a pressure exerted on the vessel wall and protecting the vessel wall. In some examples, the increased surface area of expandable support frame 124 is further configured to increase friction between neuromodulation assembly 112 and the vessel wall, thereby helping to retain neuromodulation assembly 112 in place relative to the vessel wall during the procedure.

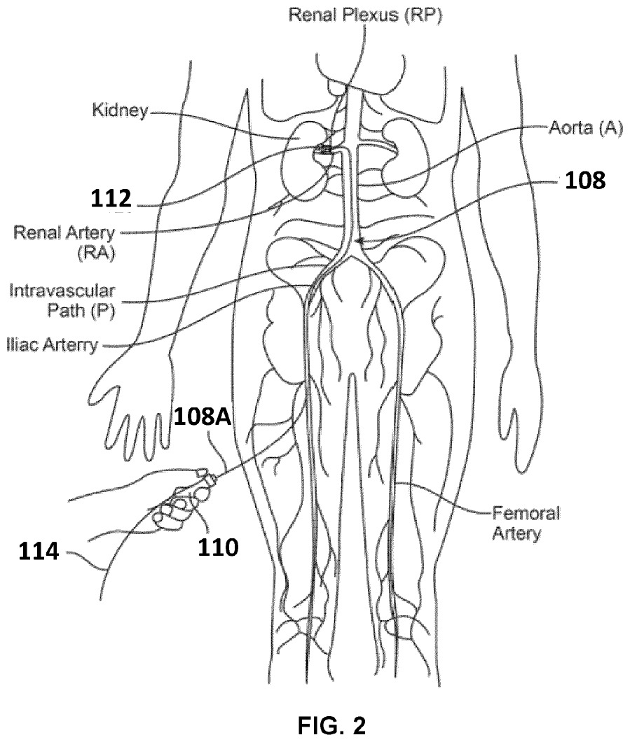

FIG. 2 (with additional reference to FIG. 1) conceptually illustrates an example technique for gaining access to renal nerves of a patient in accordance with some examples of the present technology. Neuromodulation catheter 102 provides access to the renal plexus RP through an intravascular path P, such as a percutaneous access site in the femoral (as illustrated in FIG. 2), brachial, radial, or axillary artery to a targeted treatment site within a respective renal artery RA. By manipulating proximal portion 108A of elongated shaft 108 from outside the intravascular path P, a clinician may advance at least distal portion 108B of elongated shaft 108 through the potentially tortuous intravascular path P and remotely manipulate distal portion 108B (FIG. 1) of elongated shaft 108.

In the example illustrated in FIG. 2, therapeutic elements 110 are delivered intravascularly to the treatment site using a guidewire 114 in an OTW technique. As noted previously, the neuromodulation assembly 112 at distal shaft portion 108B may define a passageway for receiving guidewire 114 for delivery of the neuromodulation catheter 102 using either an OTW or a RX technique. At the treatment site, guidewire 114 can be at least partially withdrawn or removed, and therapeutic elements 110 can transform or otherwise be moved to a deployed arrangement for delivering a chemical agent. In other examples, therapeutic elements 110 may be delivered to the treatment site within a different guide device, such as a guide sheath (not shown), with or without using guidewire 114. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the treatment site without the aid of guidewire 114 and/or a guide sheath.

An imaging device may enable image guidance, e.g., computed tomography (CT). fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal shaft portion 108B and therapeutic elements 110. For example, a fluoroscopy system (e.g., including a flat-panel detector, x-ray, or C-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic elements 110. Further, in some examples, image guidance components (e.g., IVUS, OCT) may be integrated with neuromodulation catheter 102 and/or run in parallel with neuromodulation catheter 102 to provide image guidance during positioning of therapeutic elements 110. For example, image guidance components (e.g., IVUS or OCT) can be coupled to therapeutic elements 110 to provide three-dimensional images of the vasculature proximate the target site to facilitate positioning or deploying therapeutic elements 110 within the target renal blood vessel.

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally induced, chemically induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery and relative to other physiological structures (such as an accessory renal artery), delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up-and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

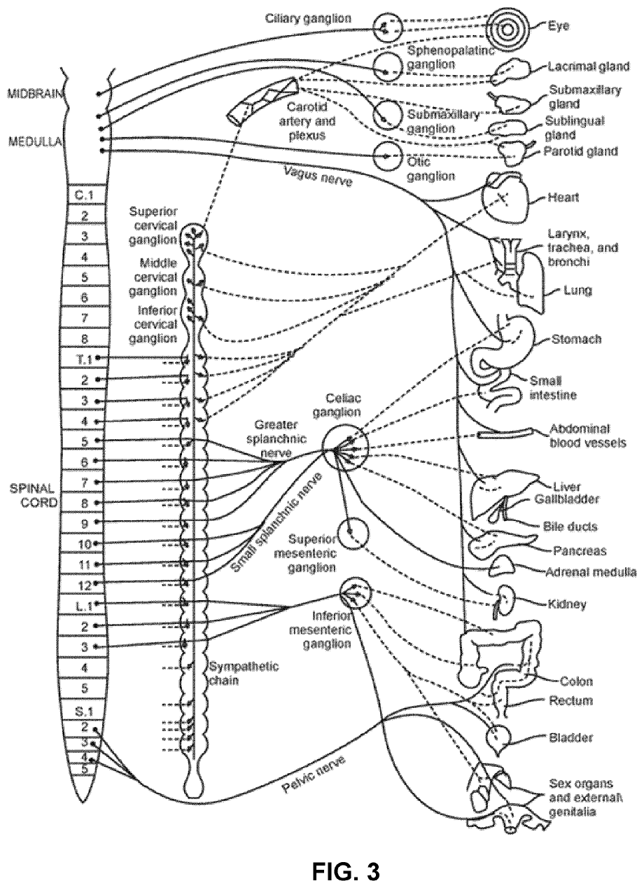

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle and inferior), which sends sympathetic nerve fibers to the head and thorax organs, and the celiac and mesenteric ganglia (which send sympathetic fibers to the gut).

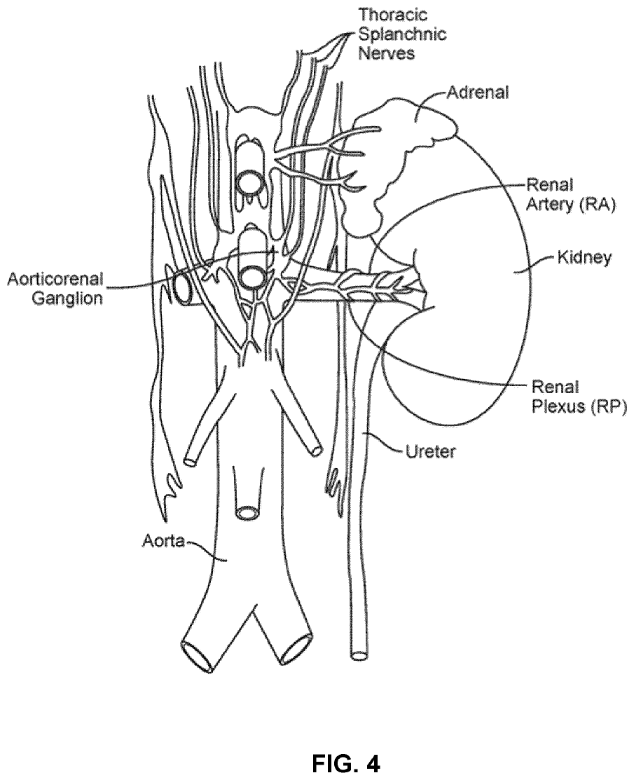

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease in some patients are characterized by heightened sympathetic nervous activation. In patients with end stage renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus and the renal tubules. Stimulation of the renal sympathetic nerves causes increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects and others.

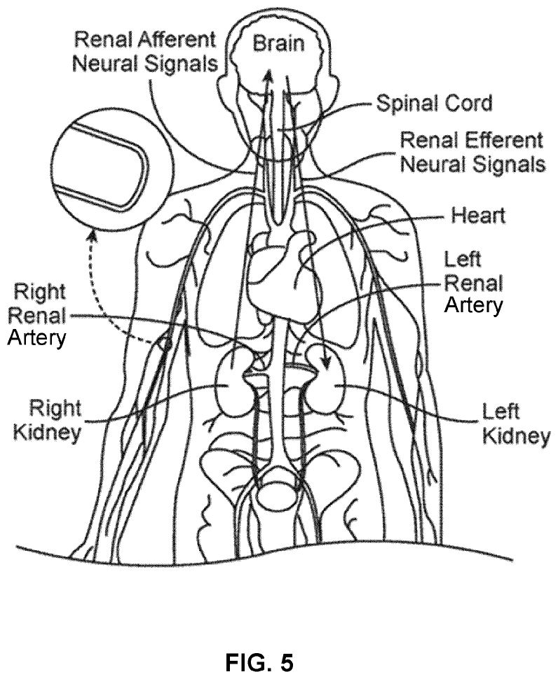

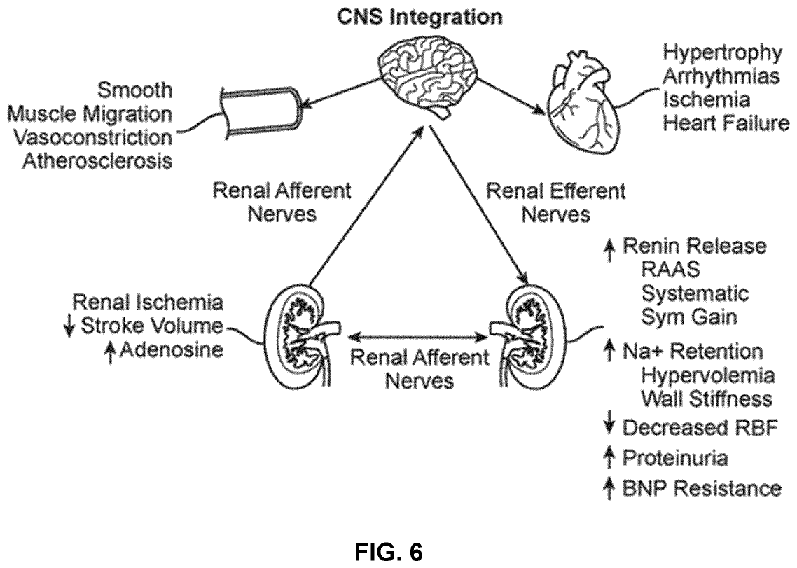

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also affects other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

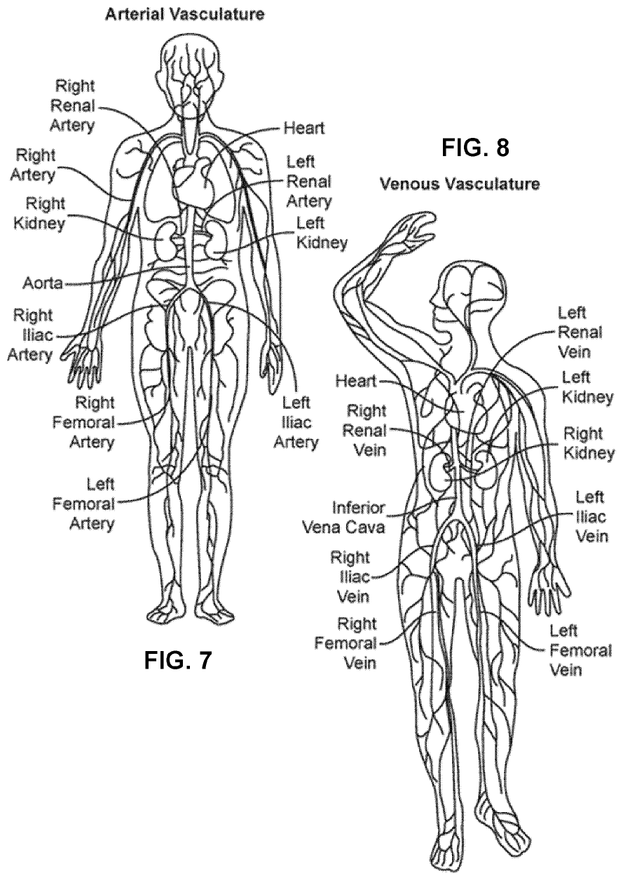

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

In some examples, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder regions provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventitia of the artery should be safely modulated via the neuromodulatory apparatus. As discussed in greater detail below, the intima-media thickness separating the vessel lumen from its adventitia means that target renal nerves may be multiple millimeters distant from the luminal surface of the artery. The chemical agent should be delivered to the target renal nerves to modulate the target renal nerves without excessively adversely affecting the vessel wall.

The neuromodulatory apparatus 112 may also be configured to allow for adjustable positioning and repositioning of the therapeutic elements 110 (FIG. 1) within the renal artery since location of treatment may also affect clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 may intimately contact the vessel wall and/or extend at least partially through the vessel wall. A renal artery vessel diameter D_{RA} is typically in a range of about 2-10 millimeters (mm), with most of the patient population having a D_{RA} of about 4 mm to about 8 mm and an average of about 6 mm. A renal artery vessel length L_{RA}, between its ostium at the aorta/renal artery juncture and its distal branchings, is generally in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the renal artery) to avoid non-target tissue and anatomical structures such as anatomical structures of the digestive system or psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus 112 a unique balance of stiffness and flexibility to maintain contact between the therapy-delivery element 110 and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

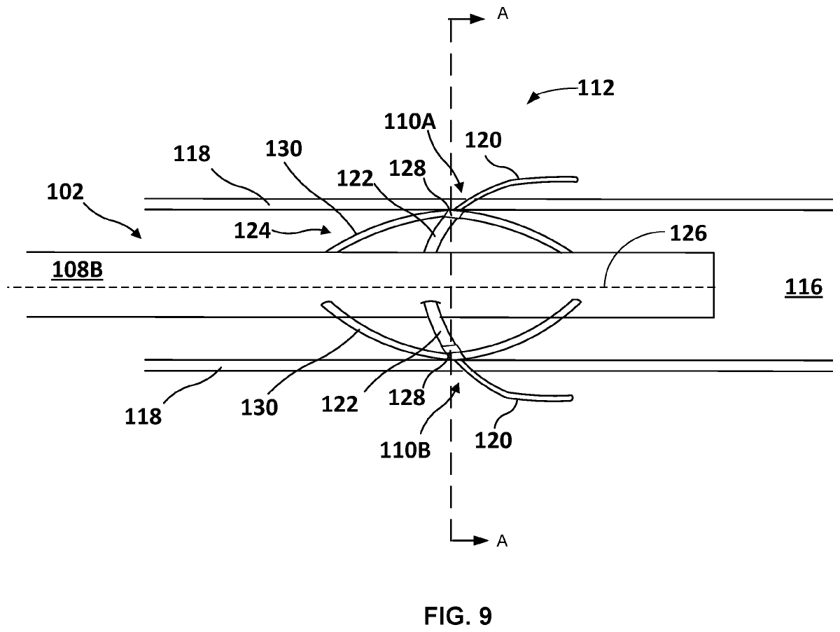

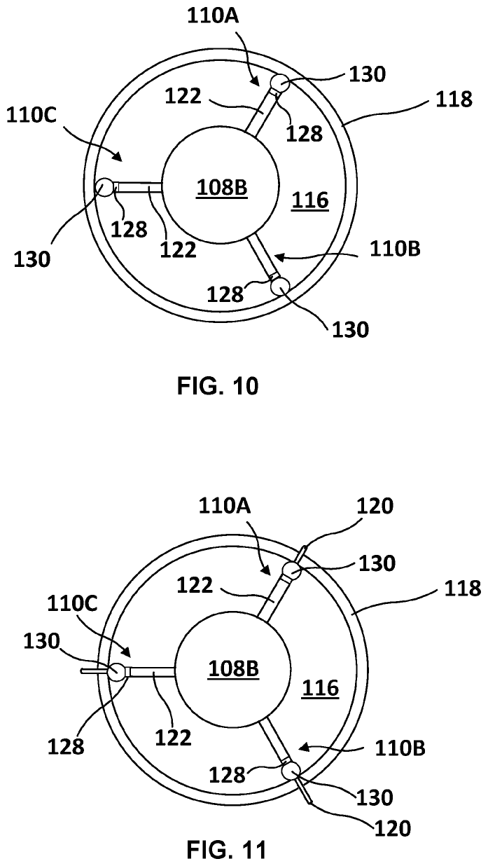

FIG. 9 is a conceptual diagram of distal shaft portion 108B of neuromodulation catheter 102 of FIG. 1 positioned within a renal artery 116 of a patient, and FIGS. 10 and 11 are cross-sectional views of FIG. 9 taken along line A-A (e.g., directly orthogonal to a longitudinal axis 126 of distal shaft portion 108B). FIGS. 9 and 11 illustrate neuromodulation assembly 112 in a “deployed” configuration in which microneedles 120 extend through a vessel wall, whereas FIG. 10 illustrates neuromodulation assembly 112 in a partially deployed configuration, in which microneedles 120 are retracted within guide tubes 122.

As shown in FIGS. 9-11, distal shaft portion 108B, including neuromodulation assembly 112, may be positioned in a distal aspect of a renal artery 116, which includes artery wall 118. Neuromodulation assembly 112 of catheter 102 includes a plurality of therapeutic elements 110 positioned circumferentially about distal shaft portion 108B. For example, therapeutic elements 110 may be spaced substantially equally around the circumference of distal shaft portion 108B. In an example with three therapeutic elements 110, the therapeutic elements 110 may be arranged at intervals of about 120°.

Therapeutic elements 110 are configured to deliver a chemical agent, such as a neurotoxic chemical, through the plurality of therapeutic elements 110. The chemical agent may be selected to neuromodulate (e.g., chemically ablate) nerve tissue of the renal plexus adjacent to renal artery 116. The chemical agent may include, for example, an alcohol, such as ethanol; distilled water; hypertonic saline; hypotonic saline; phenol; glycerol; lidocaine; bupivacaine; tetracaine; benzocaine; guanethidine; botulinum toxin; another appropriate neurotoxic fluid; or combinations thereof. In some examples, the chemical agent may be heated or cooled to additionally or alternatively thermally ablate nerve tissue of the renal plexus adjacent to renal artery 116. In other examples, the chemical agent may include a fluid configured for another purpose, such as a protective fluid, a fluid configured to increase conductivity, or fluids for other applications.

As shown in FIGS. 10 and 11, neuromodulation catheter 102 includes three therapeutic elements 110, one of which is not shown in FIG. 9 because it is occluded by the distal shaft 108B of neuromodulation catheter 102. In other examples, neuromodulation catheter 102 includes only one or two therapeutic elements 110, or more than three therapeutic elements 110. Each therapeutic element 110 includes a corresponding needle 120 and, in some, but not all, examples, a corresponding guide tube 122. Each guide tube 122 is configured to house or surround a respective needle 120 to define a pathway through which the corresponding needle 120 may traverse to reach a target site for delivery of a therapy, e.g., to reach a location of the vessel wall 118.

Therapeutic elements 110 are configured to be carried by neuromodulation catheter 102 in a “delivery” configuration as distal shaft portion 108B of neuromodulation catheter 102 is advanced through vasculature of the patient to renal artery 116. Therapeutic elements 110 are also configured to transform to a “deployed” configuration, which is shown in FIGS. 9 and 11. For example, in the delivery configuration, needles 120 may be withdrawn into guide tubes 122 and guide tubes 122 may be retracted within distal shaft portion 108B. To transition from the delivery configuration to the deployed configuration, guide tubes 122 may be advanced out of distal shaft portion 108B, extending radially outward relative to distal shaft portion 108B, and contact the wall 118 of renal artery 116, as shown in FIGS. 9, 10, and 11. After guide tubes 122 have been advanced out of neuromodulation catheter 102 to contact the wall 118, needles 120 may be advanced out of guide tubes 122 at least partially through wall 118 (e.g., only partially through wall 118 or completely through wall 118). Alternatively, needles 120 may be fixed relative to guide tubes 122, such that guide tubes 122 and needles 120 are extended out of neuromodulation catheter 102 together and the exposed length of needles 120 out of guide tubes 122 is fixed. To transition from the deployed configuration to the delivery configuration, guide tubes 122 and needles 120 may be retracted within distal shaft portion 108B.

As another example, in the delivery configuration, needles 120 may be withdrawn into guide tubes 122 and guide tubes 122 may be urged (e.g., collapsed radially inward) against an outer surface of neuromodulation catheter 102 using a guide sheath (not shown in FIG. 9). To transition to the deployed configuration, the guide sheath may be withdrawn proximally relative to neuromodulation catheter 102 (or neuromodulation catheter 102 may be advanced distally relative to the guide sheath), which allows guide tubes 122 to expand radially outward. As such, in some examples, guide tubes 122 may be formed of a resilient material, such as a shape memory material, which is self-expanding upon being released from the guide sheath. Additionally or alternatively, guide tubes 122 may include or may be formed from a radiopaque material, e.g., to facilitate medical imaging of therapeutic elements 110. For example, guide tubes 122 may be formed from a radiopaque metal, such as tantalum, gold, platinum, or the like. As another example, guide tubes 122 may be formed from a polymer and include a radiopaque marker element, such as a ring or band, formed from a radiopaque metal, such as tantalum, gold, platinum, barium, tungsten, or the like. As a further example, guide tubes 122 may be formed from a polymer filled with a radiopaque powder, such as tantalum, gold, platinum, barium, tungsten, or the like. In examples in which guide tubes 122 include a plastic, the plastic may include a polyamide, a polyurethane, or the like; or a multilayer construction that includes an inner layer of polyamide and an outer layer of polyurethane.

As shown in FIG. 9, neuromodulation catheter 102 includes a radially expandable support frame 124. In general, therapeutic elements 110 are configured to extend radially outward through support frame 124, such that the support frame 124 contacts vessel wall 118 at locations immediately surrounding the points at which therapeutic elements 110 extend outward from support frame 124. For instance, support frame 124 may define a longitudinally central portion that is approximately radially consistent across the longitudinal length of the central portion. Therapeutic elements 110 may extend radially outward through the longitudinally central portion of support frame 124, such that portions of the support frame 124, both proximal and distal to the therapeutic elements 110, contact the vessel wall 118 and distribute a contact pressure in that region. In this way, support frame 124 helps protect vessel wall 118 from a localized pressure that could otherwise result, e.g., from contact with radially outward portions 128 of guide tubes 122.

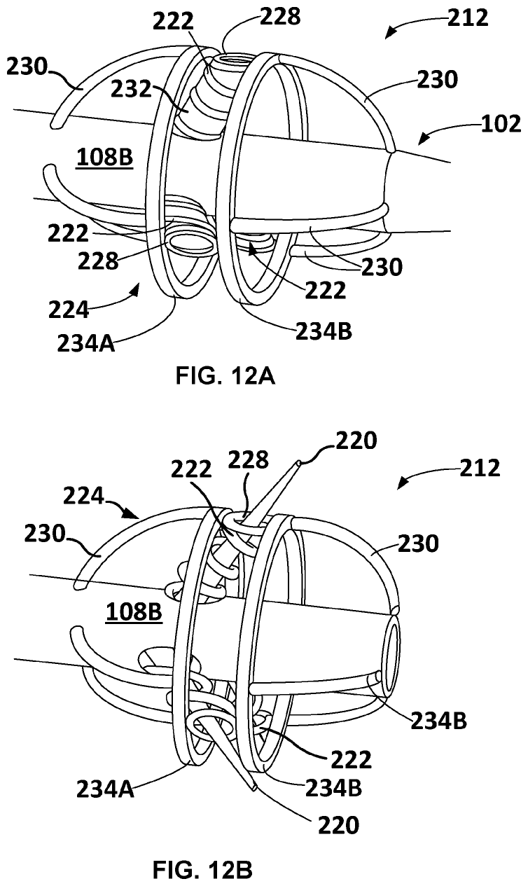

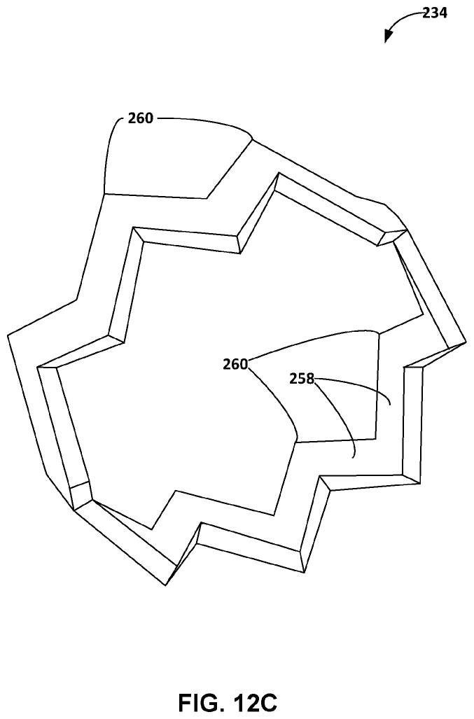

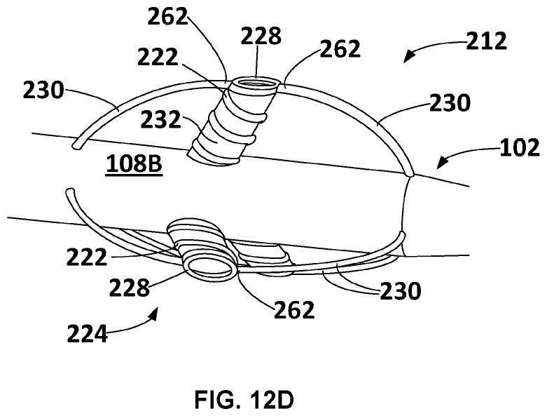

Expandable support frame 124 may be located adjacent to therapeutic elements 110, e.g., both proximally and distally. As detailed further below with respect to FIGS. 12A-16B, expandable support frame 124 may include one or more of an expandable wire basket, a cage, a stent, or the like. Expandable support frame 124 is configured to be carried by distal shaft portion 108B in a relatively low-profile delivery configuration, in which expandable support frame 124 is not expanded or is less expanded than while in a deployed configuration, and to expand to the deployed configuration after distal shaft portion 108B is positioned within renal artery 116.

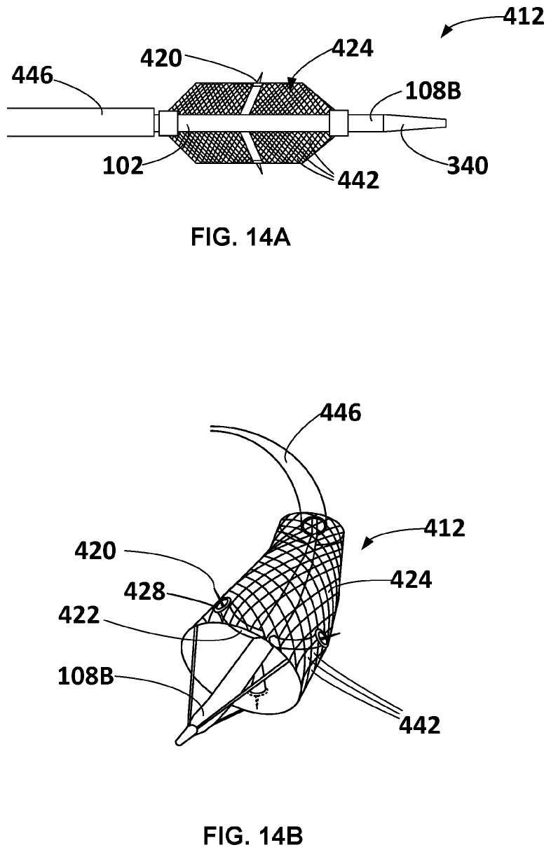

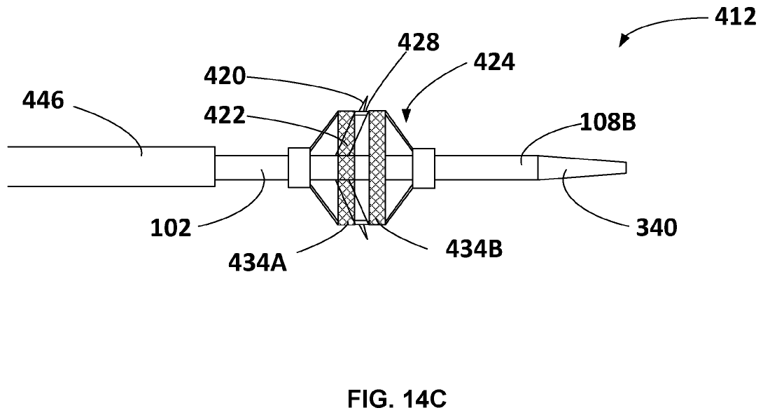

Expandable support frame 124 may be configured to expand via any suitable mechanism. For instance, expandable support frame 124 may be operatively coupled to an elongated pullwire that extends the length of catheter body 108 to an appropriate actuator mechanism, e.g., on handle 106 (FIG. 1). Additionally or alternatively, one or more components of support frame 124 may be configured to self-expand radially outward. For instance, support frame 124 may be held in a low-profile delivery configuration within an inner sheath lumen of a delivery sheath (FIG. 14A). Once extended distally outward from a distal mouth of the delivery sheath, the self-expanding components of support frame 124 (e.g., compressed springs, shape-memory-material components such as Nitinol components) may then self-expand radially outward. These same components may then collapse back down to the delivery configuration when retracted back into the distal mouth of the sheath.

As described above with respect to FIG. 1, while in the deployed configuration, at least a portion of expandable support frame 124 is configured to contact artery wall 118 to approximately center distal shaft portion 108B within renal artery 116, and to help retain distal shaft portion 108B (including neuromodulation assembly 112) in position relative to artery wall 118 during the treatment. In the example shown in FIGS. 9-11, expandable support frame 124 includes a plurality of elongated arms 130, oriented generally longitudinally (e.g., parallel to longitudinal axis 126. In some such examples, but not all examples, each one of longitudinal arms 130 is mechanically coupled to a radially outward portion 128 of a respective guide tube 122 (“outer tube portions 128”). For instance, outer tube portions 128 may be mechanically coupled to an interior surface of longitudinal arms 130, integrated within an interior portion of longitudinal arms 130, or even extend radially through longitudinal arms 130 up to a point at which an outermost surface of guide tubes 122 is co-planar with an exterior surface of longitudinal arms 130. In such examples, expandable support frame 124 is configured to protect vessel wall 118 from contact with outer tube portions 128, e.g., by redistributing an applied pressure from outer tube portions 128 across the greater surface area of longitudinal arms 130 (or an equivalent structure of support frame 124, as detailed further below).

While FIGS. 9-11 conceptually illustrate the functionality of expandable support frame 124, the particular structure of neuromodulation assembly 112 depicted in FIGS. 9-11, including support frame 124 and/or guide tubes 122, is not intended to be limiting. Radially expandable support frame 124 may include any of a number of different structures, as detailed below with respect to FIGS. 12A 16B. It is to be understood that the following examples of support frame 124 are not mutually exclusive, and elements of each example may be used in combination and/or interchangeably.

FIGS. 12A and 12B are perspective views of a neuromodulation assembly 212, which is an example of neuromodulation assembly 112 of catheter 102 of FIGS. 1 and 9-11, except for the differences noted herein. Neuromodulation assembly 212 includes guide tubes 222 (e.g., guide tubes 122 of FIGS. 9-11) and radially expandable support frame 224 (e.g., support frame 124 of FIGS. 1 and 9-11) positioned at a distal shaft portion 108B of catheter 102.

As shown in FIGS. 12A and 12B, each guide tube 222 includes a coiled or spring-like structure that elongates or extends radially outward from the catheter to surround a respective fluid-delivery needle 220 (e.g., needle 120 of FIGS. 9 and 11). In some examples, the coiled structures of guide tubes 222 may be formed from a shape-memory-material, such as Nitinol, to further enhance the configuration-transitional properties of guide tubes 222, in addition to the spring-like structure. As shown in FIGS. 12A and 12D (but not FIG. 12B), each guide tube 222 may further include a flexible layer 232, such as a polymer membrane, positioned either within or overtop of a circumference of the coiled guide tubes 222, in order to more fully encapsulate a portion of the fluid-delivery needles 220. In other examples, each guide tube 222 may be formed entirely from this type of a flexible membrane 232, e.g., without a coiled structure.

In some examples, a radially outer tube portion 228 (e.g., outer tube portions 128 of FIGS. 9-11) of each guide tube 222 is mechanically coupled to radially expandable support frame 224. In the example shown in FIGS. 12A and 12B, radially expandable support frame 224 comprises a pair of proximal and distal rings 234A, 234B, respectively (“rings 234”), and a plurality of support struts 230 (e.g., elongated legs 130 of FIGS. 9-11) extending between the proximal and distal rings 230 and the distal catheter portion 108B. In some examples, but not all examples, rings 234 and/or support struts 230 may be formed from a shape-memory material, such as Nitinol, so as to be able to self-expand from the low-profile delivery configuration to the deployed configuration show in FIGS. 12A and 12B. Additionally or alternatively, the catheter-adjacent ends of support struts 230 may be configured to extend through an opening defined by the outer surface of catheter 102, such that struts 230 may be received within an inner lumen of catheter 102 to cause neuromodulation assembly 212 to collapse radially inward into the delivery configuration.

Proximal and distal rings 234A, 234B are positioned on longitudinally opposite sides of the guide tubes 222, respectively. Collectively, rings 234 define a longitudinally central portion of support frame 224 that is approximately radially consistent (e.g., along an exterior surface of rings 234). Similar to the example described above with respect to FIG. 9, rings 234 are configured to contact vessel wall 118 (FIG. 9) and redistribute an applied pressure at locations both proximal and distal to the points at which needles 220 extend radially outward from support frame 224 (e.g., between rings 234A, 234B). In this way, rings 234 protect the vessel wall from a more focused force and higher pressure that could otherwise result from direct contact with radially outward portion 228 of guide tubes 222.

Rings 234 may be configured to expand and collapse via any suitable mechanism. For example, rings 234 may define a plurality of circumferential folds or pleats that guide or enable rings 234 to collapse circumferentially inward toward catheter 102. Additionally or alternatively, rings 234 may each include a planar coil structure, configured to self-expand circumferentially outward, e.g., when no longer retained within a lumen of a delivery sheath. In other examples, rings 234 may each include a planar coil structure configured (e.g., biased) to self-collapse circumferentially inward. For instance, rings 234 may be in a default state when in the low-profile delivery configuration. A clinician may actuate a proximal actuator (e.g., pullwire, pushwire, etc.) to cause struts 230 to expand radially outward away from catheter 102, thereby forcing rings 234 circumferentially outward and retaining rings 234 in the deployed configuration shown in FIGS. 12A and 12B. Upon “unlocking” the actuator mechanism, rings 234 may collapse circumferentially inward (e.g., radially inward, from the perspective of catheter 102), causing struts 230 to collapse radially inward as well.

In some examples, proximal and/or distal rings 234A, 234B may include an expandable-stent-like expansion mechanism. For instance, FIG. 12C is a perspective view of an example stent-like ring structure 234 formed from a plurality of struts 258 interconnected by a corresponding plurality of crowns 260. When actuated, adjacent struts 258 are configured to re-orient away from one another, causing ring structure 234 to expand radially outward from a contracted configuration to an expanded deployed configuration.

In some examples, elongated arms 230 and/or rings 234 may include or may be formed from one or more elongated filaments or filars formed from a shape-memory material, such as Nitinol. For instance, an elongated shape-memory filar may be advanced outward from a sidewall of catheter 102, or alternatively, from a distal opening to a lumen defined by a more-rigid tubular structure of one of support arms 230. The elongated filar may then be actuated to assume its predefined (e.g., heat-set) shape. For example, a distal portion of the elongated filar may be configured to convert from a substantially linear shape into a substantially circular or coiled shape, forming a structure similar to rings 234.

In other examples, such as the example illustrated in FIG. 12D, neuromodulation assembly 212 does not include either of rings 234A, 234B, and instead, support arms 230 are coupled directly to radially outermost portions 228 of respective coiled guide tubes 222. In some such examples, support arms 230 define contact points 262, e.g., located immediately proximal and distal to radially outward guide tube portions 228. Contact points 262 of support arms collectively define a longitudinally central portion of support frame 224, e.g., and are configured to contact the vessel wall to redistribute an applied pressure in the region surrounding (e.g., proximal and distal to) radially outward guide tube portions 228. Although substantially straight support arms (parallel to a longitudinal axis of catheter 102) are shown in FIG. 12D, in other examples, support arms 230 may have other shapes, such as v-shaped (with the vertex connected to coiled guide tubes 222 and the two ends of the “v” attached to the body of catheter 102), y-shaped (with the single leg connected to coiled guide tubes 222 and the two ends of the “y” attached to the body of catheter 102), or the like.

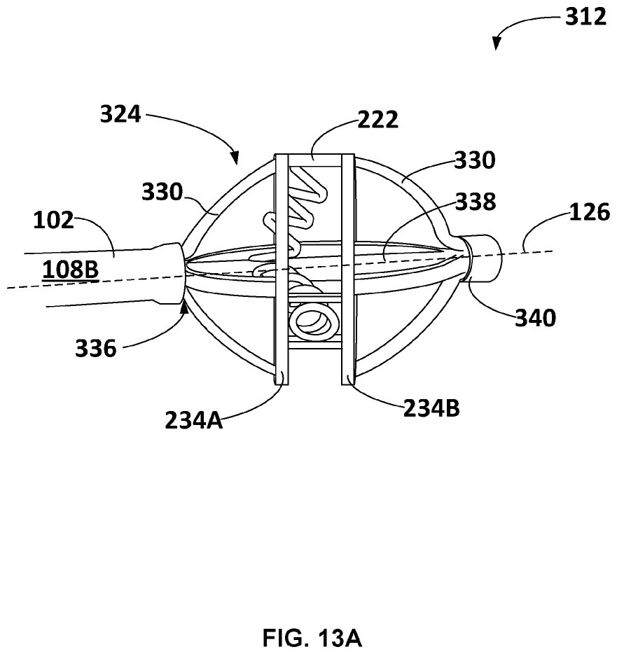

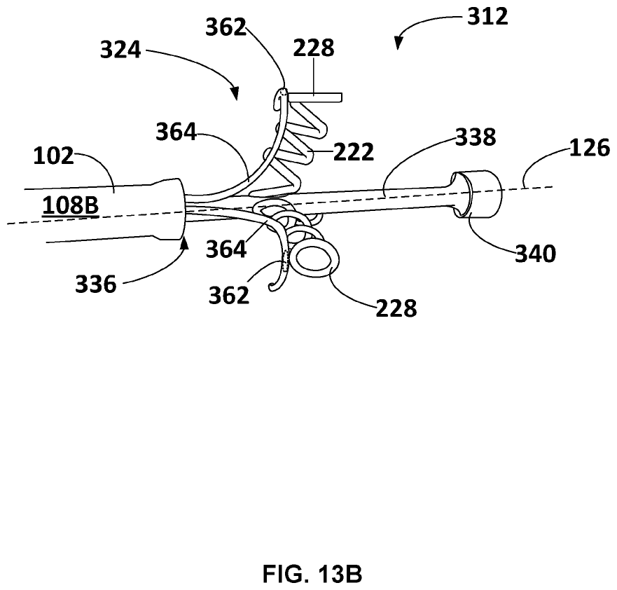

FIG. 13A is a perspective view of neuromodulation assembly 312, which is an example of neuromodulation assembly 112 of catheter 102 of FIGS. 1 and 9-11, except for the differences noted herein. In particular, neuromodulation assembly 312 is an example of neuromodulation assembly 212 of FIGS. 12A and 12B, except for the differences noted herein. For instance, rather than being positioned overtop of (e.g., radially outward from) a distal portion of distal catheter portion 108B, neuromodulation element 312 is configured to extend distally outward from a distal mouth 336 of catheter 102.

Similar to neuromodulation assembly 212, neuromodulation assembly 312 includes an expandable support frame 324 that includes proximal and distal rings 234A, 234B, positioned on either longitudinal side of coiled guide tubes 222. However, in place of more-rigid support struts 230, support frame 324 includes a plurality of more-flexible support struts 330 (e.g., longitudinal legs 130 of FIGS. 9-11) extending between rings 234 and a central elongated member. Support struts 330 are configured to bend or curve when expanding radially outward to the deployed configuration, such that, when deployed, support struts 330 collectively define circular or rounded hoops, defining respective planes oriented parallel to the central longitudinal axis 126 of catheter 102. In some such examples (but not all such examples), support frame 324 may be configured to expand radially outward according to an umbrella-type mechanism, in which an actuator mechanism causes rings 234 and struts 330 to translate distally forward as well as expand radially outward. Further, in some examples, similar to the description in FIGS. 12A 12D, rings 234 may include a single-or multiple-row stent-like structure, and/or struts 330 may have other shapes.

As shown in FIGS. 13A and 13B, in some examples, neuromodulation assembly 312 (or catheter 102, as appropriate) includes an atraumatic distal tip 340, such as a curved, flattened, and/or softer deformable structure for improved navigability through tortuous vessels of the patient. For instance, expandable support frame 324 and distal tip 340 are movable relative to a more proximal portion of neuromodulation element 312, e.g., nearer distal catheter mouth 336 in the configuration depicted in FIGS. 13A. In such examples, a proximal motion of distal tip 340, e.g., via a pullwire coupled to central shaft 338, may be configured to expand support frame 324 to the deployed configuration via an umbrella-type mechanism, whereas a distal motion of distal tip 340, e.g., via a pushwire coupled to central shaft 338, may be configured to collapse support frame 324 to the delivery configuration.

In some examples, such as the example illustrated in FIG. 13B, support frame 324 does not include circumferentially continuous rings 234. Instead, support frame 324 includes one or more elongated filaments or filars 364, either pre-shaped into a desired expanded-support-frame configuration, or formed from a shape-memory material, such as Nitinol, and configured to assume a desired expanded-support-frame configuration when actuated. For instance, elongated shape-memory filars 364 may be advanced radially and distally outward from a sidewall of catheter 102, distally outward from distal catheter mouth 336, and/or distally outward from distal openings to lumens defined by more-rigid tubular structures of support arms 330 (FIG. 13A). The elongated filars 364 may then be actuated to assume predefined (e.g., heat-set) shapes. For example, a distal portion of the elongated filars 364 may be configured to convert from a substantially linear shape into a substantially curved, rounded, or coiled shape, thereby forming a structure similar to at least a circumferential portion of rings 234 of FIG. 13A, e.g., defining radially outward contact points 362 for contacting a vessel wall.

FIGS. 14A and 14B are perspective views of neuromodulation assembly 412, which is an example of neuromodulation assembly 112 of catheter 102 of FIGS. 1 and 9-11, except for the differences noted herein. For instance, in the previous examples, expandable support frames 124, 224, 324 each included a relatively “minimal” structural frame, e.g., including only a few elongated legs, struts, and/or rings. By comparison, neuromodulation assembly 412 of FIGS. 14A and 14B includes an expandable support frame 424 with a more comprehensive or intricate structure, such as a mesh-like or stent-type frame. More specifically, support frame 424 includes an elongated tubular body that defines a plurality of openings 442. In the deployed configuration, fluid-delivery needles 420 are configured to extend radially outward via guide tubes 422 through the openings 442 defined by the tubular body.

Outer tube portions 428 (e.g., outer tube portions 128 of FIGS. 9-11) may be adhered, such as welded, glued, or otherwise mechanically coupled, to an interior surface of the tubular body of support frame 424. In some examples, such as the example depicted in FIG. 14A (but not FIG. 14B), the tubular body defines tapered proximal and/or distal portions 444A, 444B, respectively, to facilitate insertion of support frame 424 into an inner lumen of a retractable sheath 446, as described above.

In some examples, such as the example illustrated in FIG. 14C, the elongated tubular structure of support frame 424 may be longitudinally condensed into one or more stent-like rings 434A, 434B positioned on either longitudinal side of a radially outermost portion 428 of guide tubes 422. In such configurations, support frame 424 may be an example of support frame 224 of FIGS. 12A and 12B, e.g., such that rings 434A, 434B may be examples of stent-like ring 234 of FIG. 12C.

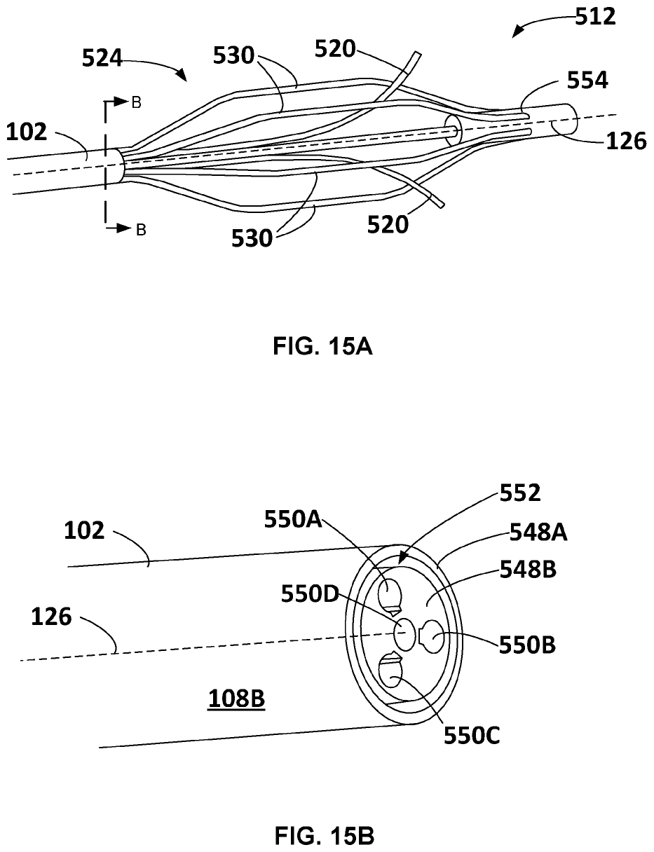

FIG. 15A is a perspective view of neuromodulation assembly 512, which is an example of neuromodulation assembly 112 of catheter 102 of FIGS. 1 and 9-11, except for the differences noted herein. FIG. 15B is a cross-sectional view of a portion of catheter 102, taken through line B-B of FIG. 15A. As shown in FIG. 15B, in some examples, at least a portion of catheter 102 includes both an outer shaft 548A, and an inner shaft 548B configured to fit within an inner lumen 552 of the outer shaft 548A. For instance, when received within outer shaft lumen 552, inner shaft 548B may be longitudinally slidable relative to outer shaft 548A. Inner shaft 548B includes a multi-lumen shaft, defining a plurality of lumens 550A-550C (“lumens 550”). Each lumen 550 is configured to receive and guide one of micro-needles 520 (e.g., needles 120 of FIGS. 9 and 11).

As shown in FIG. 15A, similar to support frame 124 of FIG. 9, support frame 524 includes a plurality of elongated struts 530 (e.g., legs 130 of FIG. 9) oriented generally longitudinally, e.g., parallel to longitudinal axis 126. In the example shown in FIG. 15A, distal ends of elongated legs 530 are rigidly coupled to a distal element 554. The mechanical couplings or configurations of the proximal ends of elongated legs 530 depends on the particular expansion mechanism of support frame 524.

For instance, in a first example “umbrella-like” expansion mechanism of support frame 524, the proximal ends of longitudinal struts 530 are rigidly coupled to outer shaft 548A, such that a distal motion of outer shaft 548A relative to inner shaft 548B is configured to cause the plurality of longitudinal struts 530 to expand radially outward into the deployed configuration of expandable support frame 524 shown in FIG. 15A. Conversely, a proximal motion of outer shaft 548A relative to inner shaft 548B is configured to cause the plurality of struts 530 to collapse radially inward to the delivery configuration of expandable support frame 524.

Similarly, in a second example expansion mechanism of support frame 524, distal element 554 may be rigidly coupled to a central elongated member 538 (e.g., central elongated member 338 of FIG. 13A). Elongated member 538 may extend proximally through a central lumen 556 of inner shaft 548B, and function as a pullwire to cause distal element 554 to move proximally relative to both inner and outer shafts 548A, 548B, respectively, thereby causing longitudinal struts 530 to deform radially outward in a mechanism similar to the first example. Conversely, a distal motion of distal element 554 relative to both inner and outer shafts 548A, 548B, may cause longitudinal struts 530 to deform radially inward to the delivery configuration.

In a third example “retractable-sheath-like” expansion mechanism of support frame 524, the proximal ends of longitudinal struts 530 are received within outer shaft lumen 552, e.g., positioned in the annular space between the interior surface of outer shaft 548A and the exterior surface of inner shaft 548B. In such examples, a proximal “retraction” motion of outer shaft 548A relative to inner shaft 548B enables the plurality of longitudinal struts 530 to self-expand radially outward into the expanded configuration of support frame 524. Conversely, outer shaft 548A may be moved distally overtop of inner shaft 548B to collapse longitudinal struts 530 back into the delivery configuration of support frame 524.

In the example shown in FIG. 15B, lumens 550A-550C each include a recess, e.g., a groove, channel, slot, and the like, for at least a portion of its longitudinal length. The corresponding needle 520 (or guide tube) within the lumen 550 can include a longitudinal protrusion, e.g., notch, bump, or the like, configured to mate with the recess and configured to reduce or substantially prevent the needle 520 from rotating about a longitudinal axis defined by the lumen. The protrusion may be configured to move longitudinally within the recess, e.g., to slide within the lumen 550. The lumen recess and the needle protrusion may be configured to reduce or substantially prevent therapeutic elements 520 from rotating and/or twisting when radially extended to contact and/or puncture a vessel wall.

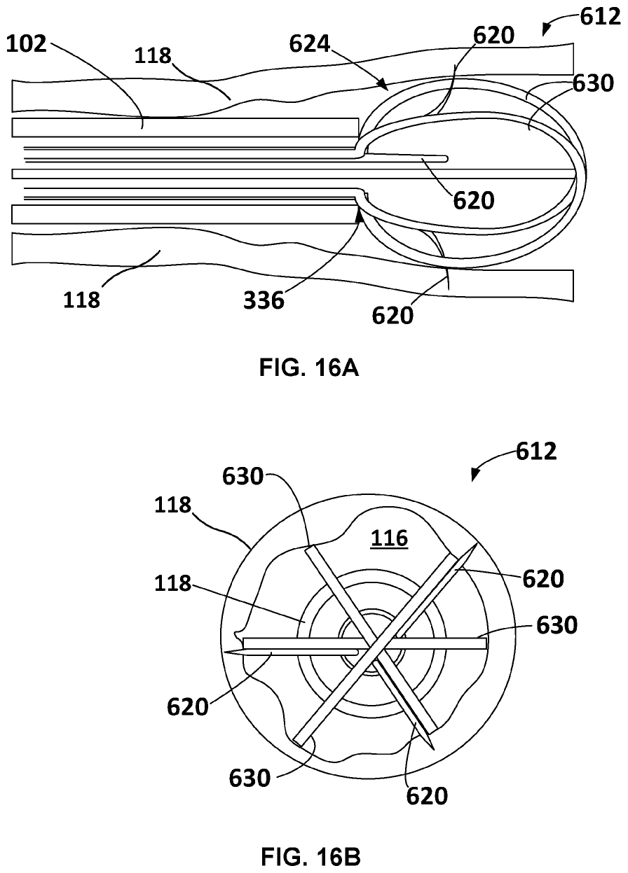

FIG. 16A is a perspective view, and FIG. 16B is an end view, of neuromodulation assembly 612, which is an example of neuromodulation assembly 112 of catheter 102 of FIGS. 1 and 9-11, except for the differences noted herein. Similar to neuromodulation element 312 of FIG. 13A, neuromodulation element 612 of FIGS. 16A and 16B is configured to extend distally outward from distal mouth 336 of catheter 102. Neuromodulation element 612 includes expandable support frame 624, which defines a “kitchen-whisk-type” configuration, formed from one or more looped wires (e.g., longitudinal legs 130 of FIGS. 9-11). As shown in FIG. 16A, each looped wire 630 bends to form a respective rounded wire hoop. Each wire hoop may be configured to self-expand radially outward when extended distally outward from distal mouth 336 of catheter 102, based on the looped shape of the wires 630 and the relative flexibility of the material forming the wires 630. In some examples, looped wires 630 may include flat wires (e.g., having a chamfered rectangular profile with the wider dimension oriented radially outward) to further distribute an applied pressure when in contact with an interior surface of vessel wall 118.

As illustrated in the end view of FIG. 16B, support frame 624 can include three looped wires 630 defining the three hoop planes, respectively. In the particular example of FIG. 16B, the three hoop planes are oriented at relative angles to one another such that the expandable support frame 624 defines a hexagonal shape formed by the six radially outermost edges of the rounded wire hoops. In some examples, fluid-delivery needles 620 may be mechanically integrated with support frame 624, such that an expansion of support frame 624 simultaneously deploys fluid-delivery needles 620 radially outward through vessel wall 118. In other examples, fluid-delivery needles 620 are operatively connected to a separate actuator mechanism (e g., via handle 106 of FIG. 1), such that they may be radially deployed separately from support frame 624.

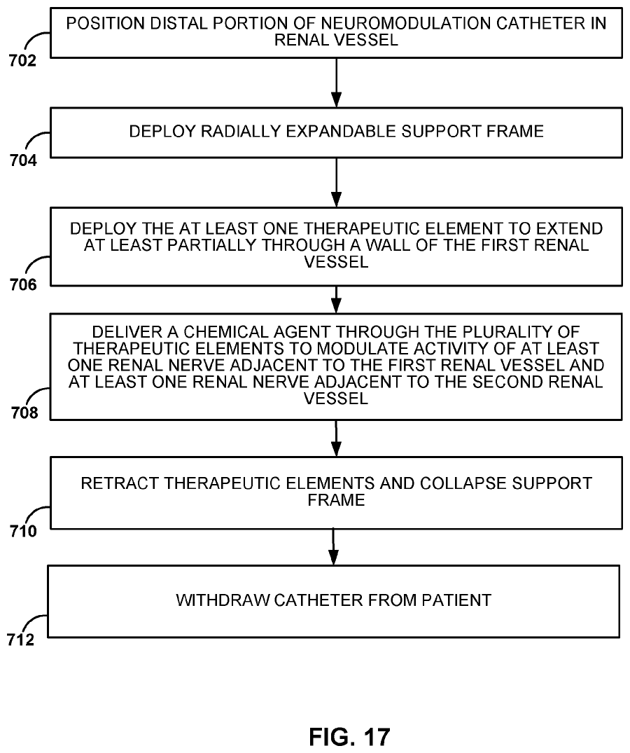

FIG. 17 is a flow diagram illustrating an example technique of performing neuromodulation of renal nerves in accordance with some examples of this disclosure. The technique of FIG. 17 will be described with concurrent reference to neuromodulation catheter 102 of FIGS. 1 and 9, although it will be appreciated that the technique of FIG. 17 may be performed with other neuromodulation catheters, such as other neuromodulation catheters described herein. Conversely, it will also be appreciated that neuromodulation catheter 102 of FIGS. 1 and 9 may be used in other techniques.

The technique of FIG. 17 includes positioning a distal portion 108B of a neuromodulation catheter 102 in a renal vessel 116 of a patient (702). A clinician may access the renal vessel 116 of the patient through an intravascular path, such as a percutaneous access site in the femoral, brachial, radial, or axillary artery to a targeted treatment site within the renal vessel 116. In some examples, the renal vessel 116 may be a main renal artery. By manipulating a proximal portion 106 of neuromodulation catheter 102 from outside the intravascular path, a clinician may advance at least distal catheter portion 108B through the potentially tortuous intravascular path and remotely manipulate distal catheter portion 108B.

Once positioned at the target treatment site, the clinician may deploy (e.g., manually actuate and/or passively enable) radially expandable support frame 124 of a neuromodulation assembly 112 positioned at the distal portion 108B of catheter 102 (704). For instance, the clinician may actuate a pullwire, engage a push mechanism, withdraw a retractable sheath, or perform any other appropriate action to cause or enable support frame 124 to expand radially outward.

The expanded support frame 124 contacts the renal vessel wall 118 at multiple locations to approximately radially center catheter 102 (and neuromodulation assembly 112) within renal vessel 116. Once expandable support frame 124 is positioned within renal artery 116, the clinician may deploy the plurality of therapeutic elements 110 to extend radially outward through expandable support frame 124 and at least partially through renal vessel wall 118 (706). In examples in which therapeutic elements 110 include both fluid-delivery needles 120 and respective guide tubes 122, expandable support frame 124 helps protect vessel wall 118 from excessive pressure applied by outer portions 128 of guide tubes 122, but distributing the applied pressure across the greater outer surface area of the support frame 124.

Once the plurality of therapeutic elements 110 have been deployed to extend at least partially through wall 118 of renal artery 116 (706), the clinician may deliver a chemical agent (e.g., an ablation fluid) through the plurality of therapeutic elements 110 to modulate activity of at least one renal nerve adjacent to renal artery 116 (708). Neuromodulation assembly 112 may then be returned to a collapsed “delivery” configuration, e.g., by retracting needles 120 through guide tubes 122 and by actuating guide tubes 122 and support frame 124 to collapse radially inward toward distal catheter portion 108B (710). The clinician may then proximally withdraw neuromodulation catheter 102 from the patient's vasculature (712).

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on delivering chemical agents, the present technology can equally be applied to other methods of neuromodulation therapy, including cooling, heating, electrical stimulation (using needle electrodes), RF energy delivery (using needle electrodes), microwave energy delivery (using microwave needles), ultrasound (using ultrasound transducers), or the like.

Certain aspects of the present disclosure described in the context of particular examples may be combined or eliminated in other embodiments. For example, any sub-components of the expandable support frames 124, 224, 324, 424, 524, and 624 may be combined in any suitable manner. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Further disclosed herein is the subject-matter of the following clauses:
1. A medical system for delivering a chemical agent into a perivasculature of a patient, the medical system comprising:
   a catheter defining a central longitudinal axis;
   one or more fluid-delivery needles configured to extend radially outward from the central longitudinal axis at a distal portion of the catheter; and
   a support frame positioned at the distal portion of the catheter,
      wherein the support frame is configured to expand radially outward from a contracted configuration to an expanded configuration;
      wherein, while in the expanded configuration, a longitudinally central portion of an exterior surface of the support frame is configured to contact and apply pressure to a vessel wall of a vessel of the patient to approximately radially center the distal portion of the catheter within the vessel; and
      wherein the one or more fluid delivery needles are configured to extend radially outward through longitudinal positions within the central portion of the exterior surface of the support frame, such that the exterior surface of the support frame distributes the applied pressure both proximal and distal to the longitudinal positions.
2. The medical system of clause 1, wherein the neuromodulation assembly further comprises one or more guide tubes, a respective guide tube of the one or more guide tubes surrounding a corresponding fluid-delivery needle of the one or more fluid-delivery needles, wherein each respective guide tube is configured to radially expand relative to the longitudinal axis of the catheter, and wherein a radially outward portion of each guide tube is coupled to the support frame.
3. The medical system of clause 2, wherein each respective radially expandable guide tube comprises a coil that is configured to radially expand and compress relative to the longitudinal axis of the catheter.
4. The medical system of clause 3, where each radially expandable guide tube further comprises a polymer sleeve positioned along an outer or inner circumference of the coil.
5. The medical system of any of clauses 1-4, wherein the one or more fluid-delivery needles comprise a plurality of fluid-delivery needles that are longitudinally aligned along the longitudinal axis of the catheter, and wherein the central portion of the support frame comprises proximal and distal rings circumferentially surrounding the distal portion of the catheter and positioned on longitudinally opposite sides of the longitudinal positions.
6. The medical system of clause 5, wherein the support frame comprises a plurality of support struts extending between the proximal and distal rings and the catheter, wherein the support struts collectively define circular hoops oriented parallel to the central longitudinal axis of the catheter.
7. The medical system of any of clauses 1-6, wherein the support frame comprises a tubular mesh frame defining a respective plurality of openings at the longitudinal positions through which the one or more fluid-delivery needles are configured to extend.
8. The medical system of clause 7, wherein the medical system further comprises a retractable sheath defining a sheath inner lumen configured to receive the tubular mesh frame and the catheter, and wherein the tubular mesh frame comprises a tapered proximal portion configured to facilitate insertion of the elongated tube into the sheath inner lumen.
9. The medical system of clause 7 or clause 8, wherein the tubular mesh frame comprises a tapered distal portion.
10. The medical system of any of clauses 1-8, wherein the catheter comprises:
   an inner shaft comprising one or more inner shaft lumens each configured to receive a respective one of the one or more fluid-delivery needles;
   an outer shaft defining an outer shaft lumen configured to receive the inner shaft, wherein the outer shaft is longitudinally slidable relative to the inner shaft; and
   a distal element rigidly coupled to the inner shaft, wherein the radially expandable support frame comprises a plurality of longitudinal struts, and wherein a distal end of each of the longitudinal struts is rigidly coupled to the distal element.
11. The medical system of clause 10, wherein a proximal end of each of the longitudinal struts is rigidly coupled to the outer shaft, wherein a distal motion of the outer shaft relative to the inner shaft is configured to cause the plurality of longitudinal struts to expand radially outward into the expanded configuration of the support frame.
12. The medical system of clause 10, wherein a proximal end of each of the longitudinal struts is received within the outer shaft lumen, and wherein a proximal motion of the outer shaft relative to the inner shaft is configured to enable the plurality of longitudinal struts to self-expand radially outward into the expanded configuration of the support frame.
13. The medical system of any of clauses 1-12, wherein the support frame comprises one or more looped wires configured to extend distally outward from a distal mouth of the catheter.
14. The medical system of clause 13, wherein the one or more looped wires comprise three looped wires each forming a respective rounded wire hoop, wherein each rounded wire hoop defines a respective hoop plane defining two radially outermost edges, and wherein the three hoop planes are oriented at angles to one another such that the radially expandable support frame defines a hexagonal shape formed by the six radially outermost edges of the rounded wire hoops.
15. The medical system of clause 13 or clause 14, wherein the one or more looped wires comprise flat or rectangular wires.
16. The medical system of any of clauses 1-15, wherein the one or more fluid-delivery needles are configured to pierce the vessel wall to deliver the chemical agent into the perivasculature.
17. The medical system of any of clauses 1-16, further comprising a pullwire configured to cause the radially expandable support frame to expand radially outward.
18. The medical system of any of clauses 1-17, further comprising a retractable sheath configured to retain the expandable support frame in a low-profile delivery configuration within an inner lumen of the retractable sheath, wherein the support frame is configured to self-expand radially outward to a deployed configuration when the support frame is extended distally outward from a distal mouth of the retractable sheath.
19. The medical system of any of clauses 1-18, wherein the catheter comprises an atraumatic distal tip.
20. A method comprising:
   introducing a catheter into a vasculature of a patient and advancing a distal portion of the catheter to a target treatment site;
   deploying a support frame of a neuromodulation assembly at the distal portion of the catheter from a contracted configuration to an expanded configuration, wherein the neuromodulation assembly comprises:
      one or more fluid-delivery needles configured to extend radially outward from a central longitudinal axis at a distal portion of the catheter; and
      the support frame, wherein, while in the expanded configuration, a longitudinally central portion of an exterior surface of the support frame is configured to contact and apply pressure to a vessel wall of a vessel of the patient to approximately radially center the distal portion of the catheter within the vessel, and wherein the one or more fluid delivery needles are configured to extend radially outward through longitudinal positions within the central portion of the exterior surface of the support frame, such that the exterior surface of the support frame distributes the applied pressure both proximal and distal to the longitudinal positions;
   deploying the one or more fluid-delivery needles through the central portion of the support frame to pierce the vessel wall; and
   introducing a chemical agent into a perivasculature of the patient via the one or more fluid-delivery needles.
21. The method of clause 20, wherein the one or more fluid-delivery needles comprise a plurality of fluid-delivery needles that are longitudinally aligned along a longitudinal axis of the catheter, and wherein the support frame comprises proximal and distal rings circumferentially surrounding the distal portion of the catheter and positioned on longitudinally opposite sides of the longitudinal positions.
22. The method of clause 21, wherein the support frame comprises a plurality of support struts extending between the proximal and distal rings and the catheter, wherein the support struts collectively define circular hoops oriented parallel to the central longitudinal axis of the catheter.
23. The method of any of clauses 20-22, wherein the radially expandable support frame comprises an elongated tubular mesh frame.
24. The method of clause 23, wherein the catheter further comprises:
   an inner shaft comprising one or more inner shaft lumens each configured to receive a respective one of the one or more fluid-delivery needles; and
   an outer shaft defining an outer shaft lumen configured to receive the inner shaft, wherein the outer shaft is longitudinally slidable relative to the inner shaft; and
   a distal element rigidly coupled to the inner shaft, wherein the support frame comprises a plurality of longitudinal struts, and wherein a distal end of each of the longitudinal struts is rigidly coupled to the distal element.
25. The method of clause 23, wherein a proximal end of each of the longitudinal struts is rigidly coupled to the outer shaft, and wherein deploying the support frame comprises actuating a distal motion of the outer shaft relative to the inner shaft to cause the plurality of longitudinal struts to expand radially outward into the expanded configuration of the support frame.
26. The method of clause 24, wherein a proximal end of each of the longitudinal struts is received within the outer shaft lumen, and wherein deploying the radially expandable support frame comprises actuating a proximal motion of the outer shaft relative to the inner shaft to enable the plurality of longitudinal struts to self-expand radially outward into the expanded configuration of the support frame.
27. The method of any of clauses 20-26, wherein the support frame comprises one or more looped wires, and wherein deploying the support frame comprises extending the one or more looped wires distally outward from a distal mouth of the catheter.
28. The method of clause 27, wherein the one or more looped wires comprise three looped wires each forming a respective rounded wire hoop, wherein each rounded wire hoop defines a respective hoop plane defining two radially outermost edges, and wherein the three hoop planes are oriented at angles to one another such that the support frame defines a hexagonal shape formed by the six radially outermost edges of the rounded wire hoops.
29. The method of any of clauses 20-28, wherein deploying the radially expandable support frame comprises actuating a pullwire configured to cause the radially expandable support frame to expand radially outward.
30. The method of any of clauses 20-29, wherein deploying the radially expandable support frame comprises extending the support frame distally outward from a distal mouth of a retractable sheath enabling the support frame to self-expand radially outward to a deployed configuration.
31. The method of any of clauses 20-30, wherein deploying the radially expandable support frame further comprises deploying one or more guide tubes coupled to the central portion of the support frame, each guide tube of the one or more guide tubes defining a pathway for a corresponding fluid-delivery needle of the one or more fluid-delivery needles.
32. The medical system of clause 31, wherein each respective radially expandable guide tube comprises a coil that is configured to radially expand and compress relative to the central longitudinal axis of the catheter.

1. A medical system for delivering a chemical agent into a perivasculature of a patient, the medical system comprising:
a catheter defining a central longitudinal axis;
one or more fluid-delivery needles configured to extend radially outward from the central longitudinal axis at a distal portion of the catheter; and
a support frame positioned at the distal portion of the catheter,
wherein the support frame is configured to expand radially outward from a contracted configuration to an expanded configuration;
wherein, while in the expanded configuration, a longitudinally central portion of an exterior surface of the support frame is configured to contact and apply pressure to a vessel wall of a vessel of the patient to approximately radially center the distal portion of the catheter within the vessel; and
wherein the one or more fluid delivery needles are configured to extend radially outward through longitudinal positions within the central portion of the exterior surface of the support frame, such that the exterior surface of the support frame distributes the applied pressure both proximal and distal to the longitudinal positions.

2. The medical system of claim 1, further comprising one or more guide tubes, a respective guide tube of the one or more guide tubes surrounding a corresponding fluid-delivery needle of the one or more fluid-delivery needles, wherein each respective guide tube is configured to radially expand relative to the central longitudinal axis of the catheter, and wherein a radially outward portion of each guide tube is coupled to the support frame.

3. The medical system of claim 2, wherein each respective radially expandable guide tube comprises a coil that is configured to radially expand and compress relative to the central longitudinal axis of the catheter.

4. The medical system of claim 3, where each radially expandable guide tube further comprises a polymer sleeve positioned along an outer or inner circumference of the coil.

5. The medical system of claim 1, wherein the one or more fluid-delivery needles comprise a plurality of fluid-delivery needles that are longitudinally aligned along the central longitudinal axis of the catheter, and wherein the central portion of the exterior surface of the support frame comprises proximal and distal rings circumferentially surrounding the distal portion of the catheter and positioned on longitudinally opposite sides of the longitudinal positions.

6. The medical system of claim 5, wherein the support frame comprises a plurality of support struts extending between the proximal and distal rings and the catheter, wherein the support struts collectively define circular hoops oriented parallel to the central longitudinal axis of the catheter.

7. The medical system of claim 1, wherein the support frame comprises a tubular mesh frame defining a respective plurality of openings at the longitudinal positions through which the one or more fluid-delivery needles are configured to extend.

8. The medical system of claim 7, wherein the medical system further comprises a retractable sheath defining a sheath inner lumen configured to receive the tubular mesh frame and the catheter, and wherein the tubular mesh frame comprises a tapered proximal portion configured to facilitate insertion of the elongated tube into the sheath inner lumen.

9. The medical system of claim 7, wherein the tubular mesh frame comprises a tapered distal portion.

10. The medical system of claim 1, wherein the catheter comprises:
an inner shaft comprising one or more inner shaft lumens each configured to receive a respective one of the one or more fluid-delivery needles;
an outer shaft defining an outer shaft lumen configured to receive the inner shaft, wherein the outer shaft is longitudinally slidable relative to the inner shaft; and
a distal element rigidly coupled to the inner shaft, wherein the radially expandable support frame comprises a plurality of longitudinal struts, and wherein a distal end of each of the longitudinal struts is rigidly coupled to the distal element.

11. The medical system of claim 10, wherein a proximal end of each of the longitudinal struts is rigidly coupled to the outer shaft, wherein a distal motion of the outer shaft relative to the inner shaft is configured to cause the plurality of longitudinal struts to expand radially outward into the expanded configuration of the support frame.

12. The medical system of claim 10, wherein a proximal end of each of the longitudinal struts is received within the outer shaft lumen, and wherein a proximal motion of the outer shaft relative to the inner shaft is configured to enable the plurality of longitudinal struts to self-expand radially outward into the expanded configuration of the support frame.

13. The medical system of claim 1, wherein the support frame comprises one or more looped wires configured to extend distally outward from a distal mouth of the catheter.

14. The medical system of claim 13, wherein the one or more looped wires comprise three looped wires each forming a respective rounded wire hoop, wherein each rounded wire hoop defines a respective hoop plane defining two radially outermost edges, and wherein the three hoop planes are oriented at angles to one another such that the radially expandable support frame defines a hexagonal shape formed by the six radially outermost edges of the rounded wire hoops.

15. The medical system of claim 13, wherein the one or more looped wires comprise flat or rectangular wires.

16. The medical system of claim 1, wherein the one or more fluid-delivery needles are configured to pierce the vessel wall to deliver the chemical agent into the perivasculature.

17. The medical system of claim 1, further comprising a pullwire configured to cause the radially expandable support frame to expand radially outward.

18. The medical system of claim 1, further comprising a retractable sheath configured to retain the expandable support frame in a low-profile delivery configuration within an inner lumen of the retractable sheath, wherein the support frame is configured to self-expand radially outward to a deployed configuration when the support frame is extended distally outward from a distal mouth of the retractable sheath.

19. The medical system of claim 1, wherein the catheter comprises an atraumatic distal tip.

20. A method comprising:
introducing a catheter into a vasculature of a patient and advancing a distal portion of the catheter to a target treatment site;
deploying a support frame of a neuromodulation assembly at the distal portion of the catheter from a contracted configuration to an expanded configuration, wherein the neuromodulation assembly comprises:
one or more fluid-delivery needles configured to extend radially outward from a central longitudinal axis of the catheter at a distal portion of the catheter; and
the support frame, wherein, while in the expanded configuration, a longitudinally central portion of an exterior surface of the support frame is configured to contact and apply pressure to a vessel wall of a vessel of the patient to approximately radially center the distal portion of the catheter within the vessel, and wherein the one or more fluid delivery needles are configured to extend radially outward through longitudinal positions within the central portion of the exterior surface of the support frame, such that the exterior surface of the support frame distributes the applied pressure both proximal and distal to the longitudinal positions;
deploying the one or more fluid-delivery needles through the central portion of the support frame to pierce the vessel wall; and
introducing a chemical agent into a perivasculature of the patient via the one or more fluid-delivery needles.

21. The method of claim 20, wherein the one or more fluid-delivery needles comprise a plurality of fluid-delivery needles that are longitudinally aligned along the central longitudinal axis of the catheter, and wherein the support frame comprises proximal and distal rings circumferentially surrounding the distal portion of the catheter and positioned on longitudinally opposite sides of the longitudinal positions.
